(19) United States
(12) Patent Application Publication
(10) Pub. No.: US 2017/0333393 A1
(43) Pub. Date: Nov. 23, 2017

(54) INJECTABLE ANTIOXIDANT FORMULATION FOR INTRAVENOUS USE OF SODIUM ASCORBATE IN HIGH DOSAGE, N-ACETYL CYSTEINE, AND DEFEROXAMINE; METHOD OF ADMINISTRATION AND USE FOR PREVENTING INJURY DUE TO REPERFUSION; AND KIT

(71) Applicant: UNIVERSIDAD DE CHILE, SANTIAGO (CL)

(72) Inventors: Ramón RODRIGO SALINAS, SANTIAGO (CL); Gina SÁNCHEZ VERGARA, SANTIAGO (CL); Juan Carlos PRIETO DOMINGUEZ, SANTIAGO (CL); Rubén AGUAYO NAYLE, SANTIAGO (CL); Fernando FLORENZANO URZUA, SANTIAGO (CL); Juan Guillermo GORMAZ ARAYA, SANTIAGO (CL)

(73) Assignee: UNIVERSIDAD DE CHILE, SANTIAGO (CL)

(21) Appl. No.: 15/532,556

(22) PCT Filed: Dec. 4, 2015
(86) PCT No.: PCT/CL2015/050048

§ 371 Date: Jun. 2, 2017

(30) Foreign Application Priority Data
Dec. 5, 2014 (CL) 3326-2014

(51) Int. Cl. A61K 31/375; B65D 81/30; A61K 31/197; A61J 1/14; A61K 31/16; A61K 9/00; B65D 81/32; B65D 81/20

(52) U.S. Cl. CPC A61K 31/375; B65D 81/32; B65D 81/30; B65D 81/20; A61J 1/14; A61K 31/16; A61K 9/0019; A61K 31/197

(57) ABSTRACT

The invention refers to an injectable antioxidant formulation for intravenous use comprising sodium ascorbate in high dosage as a first antioxidant agent, N-acetyl cysteine as a second oxidant agent and deferoxamine as an iron chelating agent, plus pharmaceutically acceptable excipients. And an administration method of said formulation that results in a synergic and improved effect to prevent injury due to reperfusion in an organ. In particular, to prevent injury due to early and late reperfusion in patients diagnosed with acute myocardium infarction and indication of primary coronary angioplasty. The invention further refers to the use of the formulation to prevent injury due to reperfusion in patients with acute myocardium infarction, subjected to primary coronary angioplasty.

### FIELD OF INVENTION

The invention relates to an injectable antioxidant formulation for intravenous use that comprises sodium ascorbate in high dosage as first antioxidant agent, N-acetyl cysteine as second oxidizing agent and deferoxamine as an iron chelating agent, plus pharmaceutically acceptable excipients. And a method for administering said formulation that results in a synergic and improved effect to prevent injury due to reperfusion in an organ. In particular, the method allows to prevent injury due to early and late reperfusion in patients with a diagnosis of acute infarction of the myocardium and indication of primary coronary angioplasty. The invention further refers to the use of the formulation to prevent injury due to reperfusion in patients with acute infarction of the myocardium, subjected to primary coronary angioplasty, and kit that contains the formulation.

### BACKGROUND OF THE INVENTION

In patients with acute infarction of the myocardium, subjected to a successful primary coronary angioplasty, a restoration of the blood flow in the occluded artery occurs, which while necessary to save the life of the patient, it exacerbates importantly the injuries caused initially due to the absence of blood flow (ischemia). This new injury subsequent to the revascularization through primary angioplasty is known as: “injury due to reperfusion”, which can lead to up to a 50% of the final magnitude of an infarction process that is also responsible for the loss of cardiac function due to reperfusion.

The previous art, in general, discloses various compositions to treat ischemic injuries due to reperfusion, which involve the use of ascorbic acid or salts or derivatives thereof, for example, JPH 09301861, US 2006030620 and JPH 02111722. Furthermore, the use of ascorbic acid or its salts is also known as an infusion in coronary Stent implants, see specifically, ITRM 20090500.

Carboxylic acid derivatives have also been used to resume the normal blood flow to the heart, and to protect the same, after the injury produced in the cardiac tissue due to ischemia and reperfusion, se for example, US 19880152501.

Furthermore, compositions and pharmaceutical kits for intravascular and simultaneous administration for their components have also been used, which comprise: a contrast agent and an antioxidant agent, particularly, N-acetyl cysteine, to prevent the injury in a damaged organ, for example, a damaged organ due to ischemia of the myocardium, infarction of the myocardium, injury by reperfusion, nephropathy, etc., see WO 2008057534.

Moreover, it is known from US 2014187509, the combined use of S-adenosylmethionine, vitamin E and vitamin C or derivatives thereof, and pharmaceutically acceptable excipients in the treatment of cardiovascular dysfunction. And since the publication of U.S. Pat. No. 4,978,668, it is known that the intravenous administration of allopurinol, oxypurinol or deferoxamine to patients suffering from tissue ischemia to reduce the injury of the same, particularly in victims that nave been subjected to post resuscitation treatment, following a heart attack.

WO 2005099758 discloses a liquid bio-artificial tissue to re-establish the function of injured tissue and organ, which is injected in the organ and recovers the function significantly in two weeks. Further teaches a cell culture medium that comprises ascorbic acid or another free radical sweeper and/or antioxidant that is used in the pre-treatment of transplantable cells prior to the transplant of an organ. The pre-treatment with ascorbic acid increases the viability of the transplanted cell and the colonization in around 50 times. This invention is particularly useful for treating ischemic cardiac injury followed by myocardium infarction.

Particularly, scientific magazines have treated the effect of intravenous administration of antioxidants on their own or combined for the treatment of injuries due to reperfusion in an experimental pig model, see Curr Ther Res Clin Exp 2008, October; 69 (5): 423-39. Moreover, the effect of an intravenous vitamin C infusion to the myocardium injury of a peri-procedure in patients that have suffered effective percutaneous coronary intervention, see Can J Cardiol. 2014, January; 30(1): 96-101. Furthermore, intravenous infusions have been revealed containing ascorbic acid to improve the degree of perfusion in the myocardium during an elective percutaneous coronary intervention, see JACC Cardiovasc Interv. 2010 February; 3(2): 221-9.

Furthermore, J Pharmacol Exp Ther. 2002, May; 301(2): 543-50 teaches that the improvement obtained in the cardio protection through glutathione with ascorbic acid, in injuries due to reperfusion of the myocardium. In Cardiovasc Drugs Ther. 1995, February; 9(1): 117-23 proposes that the use of antioxidant vitamins can reduce the magnitude of an infarction following ischemia water to the myocardium/reperfusion. And Biomed Res Int 2013 teaches the molecular base of the cardio protective effect of antioxidant vitamins on myocardium infarctions.

Whereas, the Trials 2014, 15:192 publication confirms the effectiveness of the sodium ascorbate antioxidants in high dosage and vitamin E to reduce the magnitude of the myocardium infarction in patients subjected to percutaneous coronary angioplasty (PREVEC clinical trial) with a study protocol for a random double blind controlled pilot test. Meanwhile, Clin Sci (Lond) 2013, January; 124(1): 1-15 discloses the molecular mechanisms and potential clinical applications for the use of vitamin C plus n-3 fatty acids in the cardio protection against ischemia/reperfusion, and Journal of the American College of Cardiology, Vol. 62, No. 16, 2013 proposes the use of n-3 fatty acids and vitamin C and E antioxidant supplement to reduce the effect of postoperative artery fibrillation.

Even though the previous art discloses formulations that comprise sodium ascorbate and N-acetyl cysteine along with other components, see specifically, these formulations are used in the treatment of primary biliary cirrhosis symptoms.

There is only one prior study that uses a formulation comprising injectable sodium ascorbate, amongst other components, which is useful for the treatment of myocardium infarction (see Jaxa-Chamiec and cols. 2005—Antioxidant effects of combined vitamins C and E in acute myocardial infarction. The randomized, double-blind, placebo controlled, multicentre pilot Myocardial Infarction and Vitamins (MIVIT) trial). But this study presents significant differences compared to the present study, one of which, the use of mega dosage or high dosage of sodium ascorbate. Furthermore, it is supported by a very different protocol as well that excludes injury due to reperfusion derived from primary coronary angioplasty. In any case, the intervention strategy tested in this publishing makes it theoretically impossible to inhibit injury due to reperfusion, because of the limited dosage of sodium ascorbate and other variables, which are considered in the same study.

Basili et al., 2010[Intravenous Ascorbic Acid Infusion Improves Myocardial Perfusion Grade During Elective Percutaneous Coronary Intervention. J Am Coll Carsiol Intv 2010; 3:221-229] and Wang et al., 2014 [The Effect of Intravenous Vitamin C Infusion on Periprocedural Myocardial Injury for Patients Undergoing elective percutaneous coronary intervention. Can J Cardiol 2014; 30:96-101] study the effects of the intravenous administration of isolated sodium ascorbate in angioplasty. However, in both studies, the dosages used were far lower than those indicated in the present application and for objects different to infarction. The therapy consisted in a prophylactic strategy oriented to preventing the injury associated to secondary angioplasty (elective) in patients with a partial stenosis diagnosis. This diagnosis does not constitute an acute infarction of the myocardium, and therefore, does not produce injury due to reperfusion. Therefore, it is intended to avoid injury derived from invasive intervention, which differs from the injury due to reperfusion, by which a planned intervention with low dosage of ascorbate, hours before the secondary angioplasty (elective) procedure.

Therefore, the necessity of a solution to the aforementioned technical problem, is still required, in the field of the pharmaceutical industry, field of this invention, and that relates to an injectable antioxidant formulation that comprises ascorbic acid in high dosage as a first antioxidant agent, N-acetyl cysteine as a second antioxidant agent, deferoxamine as an iron chelating agent, along with pharmaceutically acceptable excipients. This formulation is a solution to be used intrahospitalily in the form of an intravenous infusion, before, during and after an angioplasty medical procedure (deflation of ball), and should prevent or reduce injury due to reperfusion in patients with acute myocardium infarction, without generating adverse events.

### BRIEF DESCRIPTION OF THE INVENTION

Injury due to reperfusion associated to acute myocardium infarction has the issue that it affects the cardiac function of patients and generates injuries associated to an increased risk of serious clinical diagnosis, such as re-infarction, strokes, arrhythmias and cardiac arrest. These conditions not only increase importantly the risk of mortality of the patient, but also, generate elevated direct and indirect cost to health systems, further to generating losses due to loss work days.

From the epidemiological point of view, the acute myocardium infarction corresponds to the first cause of death in Chile and in the world [Rodrigo y cols. 2014, Effectiveness of vitamins C and E in reducing MI size in PCA—PREVEC Trial-study protocol. Trials 29; 15:192]. Only in the United States of America has it recently been reported that an annual incidence of 715,000 events [Singh & Cohen 2014, Therapy in ST-elevation myocardial infarction: reperfusion strategies, pharmacology and stent selection Curr Treat Options Cardiovasc Med. 16(5): 302].

Until today, no standardized solution exists in the clinical practice to prevent, attenuate or treat, injuries due to reperfusion. Previous attempts have failed, and currently, there is no prevention or treatment for this clinical issue.

Controlled clinical trials have applied intravenous sodium ascorbate before an angioplasty procedure, but none of these protocols has been: 1) Used to prevent injury due to reperfusion, 2) Used for acute myocardium infarction, 3) Efficient to recover the ventricular function after infarction, 3) Based on high sodium ascorbate dosage, 4) Using before, during and after the angioplasty procedure, and 5) Based on a formulation of three active ingredients with independent and synergic functional-biological effects, both from the chemical and biological point of view, where the combination has effect both on attenuation of the injury due to early and late reperfusion.

Basili and cols. 2010 reports the use of isolated intravenous low dosage of sodium ascorbate (1 gr), with a different objective to preventing injury due to reperfusion because it is intervened with secondary angioplasty (elective) to patients without diagnosis of acute myocardium infarction.

Wang and cols. 2014 reported the use of isolated intravenous moderated dosage of sodium ascorbate (3 gr), with a different objective to preventing injury due to reperfusion because it is intervened with secondary angioplasty (elective) to patients without diagnosis of acute myocardium infarction.

In both studies; Basili and cols., 2010 and Wang and cols., 2014, patients with acute myocardium infarction were not considered, which implies that there was never total occlusion of one or more coronary arteries (physiopathology that defines the acute infarction event) therefore reperfusion could not be produced either, nor the subsequent injuries associated to this process (injury due to reperfusion), which is the object of the present application.

The present invention is related to a pharmaceutical formulation administered by infusion, which comprises ascorbic acid in high dosage as a first antioxidant agent, N-acetyl cysteine as a second antioxidant agent and deferoxamine as an iron chelating agent, this formulation is useful for preventing injuries due to reperfusion in patients with indication of primary angioplasty due to diagnosis of acute myocardium infarction.

Another embodiment of the invention is a procedure to administer this infusion before, during and after angioplasty, in order to obtain favourable results, never before reported in the prevention of injury due to reperfusion in controlled clinical trials, therefore, preventing part of the functional damage to the heart derived from this injury.

Yet another embodiment of this invention is, the use of this formulation, prepared with these three components, to prevent injury due to reperfusion in primary angioplasty procedures.

### BRIEF DESCRIPTION OF DRAWINGS

FIG. 1: Illustrates the effect of the intervention in the left ventricular ejection fraction, evaluated by means of cardiac magnetic resonance on day 6 and day 84 post-primary angioplasty. FEVI: left ventricular ejection fraction; LA group: Placebo group HA group: Intervened group.

FIG. 2: Illustrates the effect of intervention on the difference in the left ventricular ejection fraction evaluated by means of cardiac magnetic resonance on day 6 and day 84 post-primary angioplasty. FEVI: left ventricular ejection fraction; LA group: Placebo group HA group: Intervened group.

FIG. 3: Effect of the interventions with both secondary components, isolated, together and in conjunction with sodium ascorbate on the recovery of heart contractibility post-reperfusion in isolated rat hearts. NaCl: “placebo” group; DFO: deferoxamine; NAC: N-acetyl cysteine; AA: sodium ascorbate. N=3 rats per group.

### DETAILED DESCRIPTION OF THE INVENTION

The object of the invention is a pharmaceutical formulation with is antioxidant and injectable, of intravenous administration, such as an infusion, which comprises sodium ascorbate in high dosage, preferably, in an amount in the range of 30 to 60 g/L, N-acetyl cysteine, preferably, in an amount in the range of 3 to 6 g/L, and deferoxamine, preferably, in an amount in the range of 2 to 4 g/L plus pharmaceutically acceptable excipients, which is useful to prevent injury due to reperfusion in patients with myocardium infarction subjected to primary angioplasty.

Another object of the invention is the preparation and administration method of said solution, where the preparation is carried out from the pure components of pharmaceutical grade, not dissolved, or in other pharmaceutical forms, injectable through intravenous administration, which can contain each component in separate form and increased concentration.

Both strategies of formulation will give through to a product with the three components mixed; a diluted solution with the tri-association.

Previous to use, the formulation can be in the tri-associated form o can be prepared immediately before commencing the infusion. The starting time of the infusion is in the range of between 40 and 20 minutes before the angioplasty, with a flow interval corresponding to a minimum velocity of 10 mL/min and maxima of 20 mL/min to maintain during the first 60 minutes of intravenous administration.

Normally, the rechanneling of the artery is produced in a standard procedure between 20 and 30 minutes in the infusion, time which is doubled or tripled for complex procedures. After the first 60 minutes, the flow of infusion is reduced to 3 mL/min, during the following two hours.

Therefore, the formulation can comprise ascorbic acid in a concentration between 160 and 320 mmol/L, in an infusion oriented to achieve plasmatic concentrations of 10 mM or higher than the plasmatic concentrations before the angioplasty (deflation of ball); N-acetyl cysteine in a concentration between 18.4 to 36.8 mmol/L in an infusion; and deferoxamine in a concentration of 3.0 to 6.1 mmol/L in an infusion.

Currently, the PREVEC formulation, massive isolated dosages of sodium ascorbate, has only been tried under very controlled conditions and with the support of professionals hired for each study, external to the clinical routine of the angioplasty procedure for diagnosis of infarction. This implies that a stable pharmaceutical form has not been developed, susceptible of incorporating the standard clinical practice for angioplasty, which allows for routine personnel of said procedure to administer the formulation in a similar way to other intravenous medicaments of common use in these interventions.

The present procedure requires that for each patient intervened the routine personnel for the angioplasty be assisted by a specially trained nurse that prepares the formulation starting from an existing very concentrated pharmaceutical formula of sodium ascorbate for intravenous use (Pascorbin) and a standard clinical flask with bi-distilled water. The nurse then must empty 375 mL of clinically bi-distilled water from the clinical flask with a graded 500 mL laboratory test tube, to fill it, using a sterile graded syringe of 50 mL attached to a hypodermic needle of calibre 16G1, with the same volume of pascorbin, corresponding to 7 and a half flasks of said product in its commercial forma (Imported specifically for the study), work that must be carried out under laminar flow conditions to maintain sterilization. This process takes several minutes and, given the chemical instability of the new solution under aerobic and lighting conditions, it must be carried out once the patient is admitted to the assistance centre, with the most haste and diligence possible in order for him to be ready upon entry of the patient to the angioplasty OR.

The current clinical practice makes it unfeasible in the present to integrate this procedure with the standard routine of angioplasty of the health systems for different reasons. The present formulation suffers from this same negative externality with the aggravating aspect of requiring a preparation with three simultaneous components, which requires a second person, a nursing technic to assist the first professional.

For this reason that for the effects of the present application the packaging of the pharmaceutical formulation in a stable form in a ready to use container is proposed to be used in primary angioplasty and adapted to the standard routine of the clinical practice.

However, to avoid photo degradation of the sodium ascorbate, the formulation is contained in a kit that comprises a container with a visible UV filter, sealed under inert atmosphere to avoid oxidation of the sodium ascorbate and N-acetyl cysteine, sterilized beforehand to avoid microbiological contamination. The container can have two compartments in order to separate two phases; the deferoxamine, in solid state or in stabilized solution through pharmaceutical technology, of the combination of sodium ascorbate and N-acetyl cysteine in solution, allowing for an easy mixture of both phases when these are combined through energetic agitation, “breaking” of one of the compartments or “injection” of un phase into another. Moreover, the container requires sealing means that allow an easy and fast visual evaluation of the integrity of the kit, to confirm that no prior unwanted mixing of the phases has occurred, which could have degraded the formulation and to verify that the desired mixing for the activation of the formula occurred in an effective manner.

For the effects of evaluating scientifically the proposed invention, two independent experimental approximations were carried out.

The attenuation of the injury due to late reperfusion was demonstrated preliminarily with the first active component in an isolated way; sodium ascorbate in massive dosage, in the PREVEC study; PHASE II clinical trial. The attenuation of the injury due to early reperfusion, was demonstrated preliminarily with the tri-association in a murine model in an isolated heart. Said model allowed to conclude that both secondary components; N-acetyl cysteine and deferoxamine have each, separately, the capacity to attenuate the injury due to early reperfusion, effect that increases synergistically when combining both components, even in absence of sodium ascorbate.

Clinical Trial in Human Patients

The main active ingredient in the present formulation of three components; sodium ascorbate in high dosage was tested isolated in a controlled PHASE II (PREVEC) clinical trial, registering favourable results in terms of capacity of attenuating the effects of injury due to late repercussion.

In a group of 50 patients subjected to a primary angioplasty due to acute infarction diagnosis, treated only with massive dosage of sodium ascorbate (equivalent dosage to the one used in the three component formulation), a recovery was observed that has never before been reported of the cardiac function in a medium term, only referable to an attenuation of the late effects of the injury due to reperfusion. The evaluation was carried out by means of cardiac magnetic resonance, technique considered the present gold standard for this kind of studies.

Methodology for Clinical Trial in Human Patients

A randomized, double blind, placebo controlled clinical trial was applied to patients diagnosed with acute myocardium infarction with indication of primary angioplasty, previously enrolled.

As inclusion criteria, was defined: Individuals of any sex older than 18 years of age; Latency of admission of up to 12 hours since presenting symptoms; First myocardium infarction; Killip 1 and 2; Capacity and willingness to sign consent form.

As exclusion criteria was established: Renal or hepathic insufficiency history; Cardiac failure history (NYHA III, IV); Cardiogenic shock; Comorbility that determines a life expectancy<6 months; Participation in another investigation; Pregnancy; Glucose 6—Phosphate Dehydrogenase deficiency.

Patients were administered an oral dose of vitamin E (800 IU), followed by a sodium ascorbate infusion (320 mmol/L). The placebo group received an inert infusion of sodium chloride solution of the same osmolarity as the sodium ascorbate infusion. Serial blood samples were taken to analyse biomarkers related to myocardium, injury, oxidative stress and inflammation in order to evaluated efficiency on injury due to early reperfusion. Cardiac magnetic resonance exams were carried out, 6 and 84 days after the angioplasty in order to determine the size of the infarction and cardiac function, in order to evaluate efficiency on injury due to early and late reperfusion.

The statistical analysis of the preliminary results, was carried out using non parametric statistics, using the median as position measurement, interquartile range as dispersion measurement and the Mann-Whitney U test to determine the statistical significance. Differences between the multiple groups was analysed by means of a Kruskal-Wallis test. For all cases, the statistical significance was assumed for P<0.05.

Results of the Clinical Trial in Human Patients

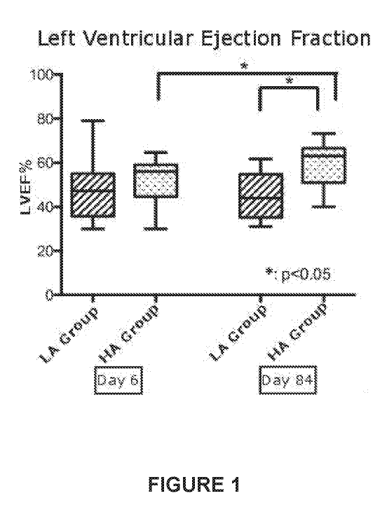

Passed three months from the moment the angioplasty took place, the cardiac function, evaluated through the left ventricular ejection fraction by means of nuclear magnetic resonance, was a 33% higher in treated patients versus placebo (p<0.05), benefit that solely referable to an attenuation of the late effects of the injury due to reperfusion (FIG. 1). The early cardiac function did not show any difference between both groups (FIG. 1).

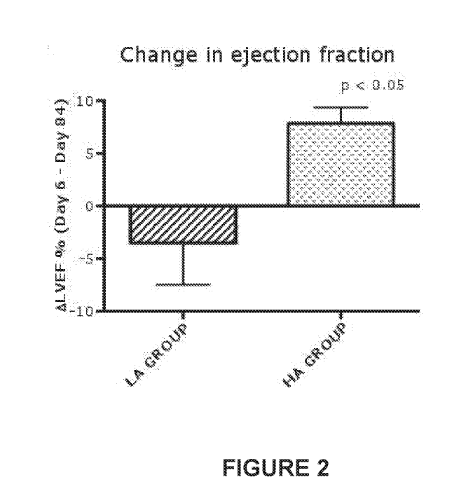

Moreover, there was an important individual change in the left ventricular ejection experienced by each patient, between both groups, which was compared by means of the calculation of the difference between the value of this variable 84 and six days after angioplasty. The change in the left ventricular ejection was shown to be significantly higher in the treated group (p<0.05), change that also showed an improvement regarding the value of the first resonance (6 days) in comparison to the placebo group, which showed a deterioration of this variable three months after the angioplasty (FIG. 2).

In Vitro Trials in Murine Model

Laboratory trials in murine model, by means of the use of the Langendorff isolated heart model, allowed to preliminarily establish the contribution of the secondary components of the solution in isolated form, in conjunction and in tri-association with sodium ascorbate on the attenuation of lethal reperfusion by means of the evaluation of the percentage of recovery of contractibility, subsequent to the reestablishment of the blood flow, after a total ischemia.

Murine Model Methodology

Sprague-Dawley adult male rats (250 to 300 g) were anesthetized with pentobarbital (80 mg/kg IP). Heparine (100 U/kg IV) was administered and the hearts were rapidly extracted and perfused with Krebs-Henseleit solution oxygenated in a Langendorff perfusion system at 37° C. [Donoso and cols., 2014. Stimulation of NOX2 in isolated hearts reversibly sensitizes RvR2 channels to activation by cytoplasmic calcium. J Mol Cell Cardiol 68:38-46]. The ventricular pressure was measured with a latex balloon inserted, in the left ventricular and connected to a pressure transductor.

After a stabilization period (15 minutes), a temporary regional ischemia of the left ventricular was produced by means of the placement of a prolene 6-0 tie under the appearance of the first anterior descendent artery (ADA) branch. The ends of the suture were joined by means of a polyethylene tube (PE-50) to form a loop for reversible occlusion. The hearts were subjected to 30 minutes of regional ischemia followed by 60 minutes of reperfusion in the presence or absence of the solution, object of the invention.

Both antioxidant agents and deferoxamine were administered during the last 10 minutes of the regional ischemia and in all the reperfusion period, both in isolated form or in association in the indicated concentrations (mmol/L): ascorbic acid: 320, N-acetyl cysteine: 36.8; deferoxamine: 3.9, “Placebo” hearts were perfused with the same concentration of antioxidants without ischemia.

Hearts were excluded if the time between the cardiac cleavage from, the thorax and the start of the perfusion through the aorta was over two minutes (longer periods of time can produce ischemic preconditioning). Also were excluded if during the stabilization period a perfusion pressure of 60 to 70 mmHg was not achieved, if the ventricular pressure developed was lower than 65 mmHg or if the cardiac frequency could not adjust to 250 to 350 beats per minute.

Evaluation of the injury: The capacity was measured of of both components isolated, in conjunction and in tri-association with sodium ascorbate in high dosage, in order to recover the cardiac contractibility, loss associated to the injury due to reperfusion.

For the analysis of the results parametric statistic of the continuous variables was used, expressing the position in terms of arithmetic mean and dispersion based on the standard deviation. The differences between multiple groups were analysed by means of ANOVA with Bonferroni test as Post-Hoc. For all results P<0.05 was considered as statistically significant.

Murine Model Results

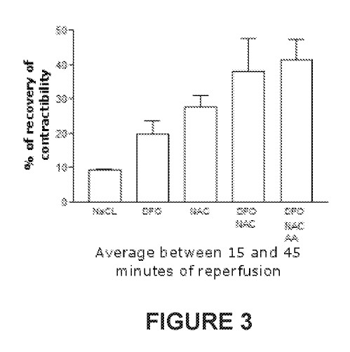

In an isolated heart murine model, a favourable effect was observed on the injury due to reperfusion of short term, using the 3 components of the solution of antioxidant infusion in separate and in conjunction. It can be appreciated that both secondary components N-acetyl cysteine and deferoxamine were capable of attenuating the injury due to early reperfusion, each separately, in conjunction and in tri-association with sodium ascorbate in high dosage, measured as a functional effect on the cardiac contractibility, further observing the synergic effect (FIG. 3). The recovery of the contractibility regarding the control group (Placebo) improved in 113% for deferoxamine, in 189% for N-acetyl cysteine and 302% for the combination of both compounds. This last value is equivalent to recovering the cardiac contractibility post-reperfusion in 40% versus the recovery near 10%, which is obtained in the untreated group.

The addition of sodium ascorbate in high dosage to the N-acetyl cysteine and deferoxamine group did not have effects on the attenuation of the injury due to early reperfusion, associated to these last two components, consistent observation with the evidence obtained from human patients, where the beneficial effects of this vitamin was seen in the attenuation of injury due to late reperfusion (FIG. 1 and FIG. 2).

Synergic Effect of Tri-Association

The present application is based on a temporary and spatial synergy of the association of three components, oriented specifically to attenuating both the early and late effects of the injury due to reperfusion. The synergic effect between the components of the solution, namely ascorbic acid, N-acetyl cysteine and deferoxamine, components that separately have shown effects, though beneficial, which do not reach the levels obtained with the tri-association.

Injury due to reperfusion can be classified in a simplified way in two stages; early injury and late injury [Seropian and cols., 2014. Anti-inflammatory strategies for ventricular remodelling following ST-segment elevation acute myocardial infarction. J Am Coll Cardiol.; 63: 1593-603]. The early injury comprises mainly a violent cellular death due to necrosis that prevails minutes after the reperfusion and is a direct consequence of the oxidative stress action. Late injury comprises a slow but sustained cellular death that occurs mainly as apoptosis as an indirect consequence of the oxidative stress and prevails from days to weeks after the reperfusion, along with a remodelling of the tissue that also influences in the loss of cardiac function. All the aforementioned mediated in an important way by the immune system.

The PREVEC intervention (only sodium ascorbate in massive dosage), is acting mainly on the injury level due to late reperfusion, presumably due to the fact that the levels of sodium ascorbate in its active form are enough to attenuate the oxidative stress to a level capable of inhibiting the activation of the immune system that, favours the apoptosis and remodelling. However, these levels of sodium ascorbate in its active form would not be capable of attenuating the oxidative stress to sufficient levels to reduce in the expected way the injury due to early reperfusion.

The animal model proves that when faced with the injury due to early reperfusion, both deferoxamine and N-acetyl cysteine, were capable of exercising each separately an independent effect of the sodium ascorbate, effect that showed synergy when both compounds are mixed.

The proposed global synergy for the tri-association considers: 1) a chemical spatial synergy between the secondary components, oriented to attenuating the injury due to early reperfusion, measured in an animal model and 2) a temporary synergy derived from the observation that the sodium ascorbate, as administered in human patients, attenuates the injury due to late reperfusion.

In other words, the present application proposes a tri-associated formula capable of attenuating synergistically the injury associated to injury due to reperfusion on a spatial level, considering the combined effect of N-acetyl cysteine and deferoxamine on the early injury and temporal level, by conditioning the aforementioned benefit the effect of the sodium ascorbate in high dosage on the late injury, strategy that has yet to be proposed.

Other procedures in which the following formulation can be applied are reperfusion therapy by pharmacological thrombolysis of the acute myocardium infarction, for the therapy of reperfusion for strokes, organ transplants and in general any other procedure that involves reperfusion of an infarction or non-infarction of a region of some organ.

1-11. (canceled)

12. A pharmaceutical injectable antioxidant composition to be administered intravenously, to a patient, before, during or after an angioplasty procedure, characterized in that it comprises ascorbic acid in high dosage or a pharmaceutically acceptable salt thereof as a first antioxidant agent N-acetyl cysteine as a second antioxidant agent and deferoxamine and further pharmaceutically acceptable excipients, useful for preventing injury due to reperfusion in said patients subjected to the angioplasty procedure.

13. A pharmaceutical composition according to claim 12, characterized in that the ascorbic acid is in effective amount in the range of 30 to 60 g/L of sodium ascorbate.

14. A pharmaceutical composition according to claim 13, characterized in that the ascorbic acid is in a concentration of between 160 to 320 mmol/L in an infusion oriented to achieving plasmatic concentrations of 10 mM or higher to the plasmatic concentrations prior to the angioplasty (deflation of ball).

15. A pharmaceutical composition according to claim 12, characterized in that the N-acetyl cysteine is in effective amounts in the range of 3 to 6 g/L.

16. A pharmaceutical composition according to claim 12, characterized in that the N-acetyl cysteine is in a concentration of between 18.4 to 36.8 mmol/L in an infusion.

17. A pharmaceutical composition according to claim 12, characterized in that the deferoxamine is in effective amounts in the range of 2 to 4 g/L.

18. A pharmaceutical composition according to claim 12, characterized in that the deferoxamine, is in a concentration of 3.0 and 6.1 mmol/L in an Infusion.

19. A procedure to prevent injury due to reperfusion before, during and after subjecting a patient to an angioplasty procedure, characterized in that it comprises administering the patient, a pharmaceutical composition as claimed in claim 12, by means of infusion in a concentration in the range of 10 to 20 mL/min, between 40 and 20 minutes before the angioplasty (deflation of ball), at a minimum velocity of 10 mL/min and maximum velocity of 20 mL/min, and/or administering said infusion during the first 60 minutes of the angioplasty procedure, at a minimum velocity of 10 mL/min and maximum velocity of 20 mL/min, the later optionally reduce said velocity to 3 mL/min, during the following two hours.

20. A pharmaceutical injectable antioxidant composition to be administered by means of infusion or intravenously, characterized in that it comprises sodium ascorbate in high dosage or another pharmaceutically acceptable salt thereof as a first antioxidant agent, N-acetyl cysteine as a second antioxidant agent and deferoxamine, and further pharmaceutically acceptable excipients, useful for treating injury due to reperfusion in pharmacological thrombolysis of acute myocardium infarction, reperfusion of stroke or a condition that comprises perfusing the infarction or non-infarction region of an organ.

21. A pharmaceutical kit, characterized in that it comprises:
a container with visible UV filter to avoid the photo-degradation of the sodium ascorbate, containing sodium ascorbate,
where the container has been sealed under an inert atmosphere in order to avoid the oxidation of the sodium ascorbate and N-acetyl cysteine,
the container has further been fabricated under sterile conditions to avoid microbiological contamination, and
has at least two compartments to separate the deferoxamine that is in solid state from the sodium ascorbate and the N-acetyl cysteine that are dissolved in liquid phase, and
has a shape that allows easy mixing of both phases, in separate compartments, when these are combined through energetic shaking, breaking of the internal compartment or injection of a phase upon the other, and further
comprises sealing means that allow an easy and fast visual evaluation of the integrity of the kit, in order to confirm that there was no previous, an unwanted mixing of the phases and also verify that mixing occurred effectively in the desired moment.
